(19) United States
(12) Patent Application Publication
(10) Pub. No.: US 2012/0088279 A1
(43) Pub. Date: Apr. 12, 2012

(54) ALGAL LIPID PRODUCTION

(75) Inventors: Andrew Keith Swanson, Cleveland Heights, OH (US); Bradley Lynn Postler, St. Charles, MO (US); Richard T. Sayre, Webster, MO (US)

(73) Assignee: Phycal, Inc., Highland Heights, OH (US)

(21) Appl. No.: 13/319,745

(22) PCT Filed: May 11, 2010
(86) PCT No.: PCT/US10/34338

§ 371 Date: Dec. 30, 2011

Related U.S. Application Data
(60) Provisional application No. 61/177,101, filed on May 11, 2009.

(51) Int. Cl. C12P 7/64; C12M 1/00; C12N 1/12

(52) U.S. Cl. 435/134; 435/257.3; 435/289.1; 435/292.1

(57) ABSTRACT

Methods for increasing the levels of lipids in oleaginous algae are described. Lipid levels in algae can be increased by stress, such as nutrient stress, after which the lipid can be harvested from the algae using a non-destructive extraction process. The stress may be provided in a periodic or “pulsed” fashion. Lipid levels in oleaginous algae can also be increased using simulated stress by treating the algae with a chemical inhibitor or by using recombinant technology to insert a sequence expressing a protein such as a nitrate reductase inhibitor that is expressed when a stressed state is desired. A method for maintaining the temperature and water levels of algae ponds using buoyant spheres is also described.

### CONTINUING APPLICATION DATA

This application claims the benefit of U.S. Provisional Application Ser. No. 61/177,101, filed May 11, 2009, the disclosure of which is incorporated by reference herein.

### BACKGROUND

The price of petroleum has fluctuated dramatically, reaching record highs of greater than US $140 per barrel in 2008. In part, those price increases reflected economic, political and supply chain uncertainties. Political concerns about the availability of petroleum supplies have led to the realization that energy independence for the US is of critical strategic importance, both economically and militarily. There also is general agreement that the release of CO₂ from fossil fuel combustion contributes substantially to global warming and climate change and must be reduced. As a result of these concerns, the domestic production of biofuels has become an increasingly attractive alternative to the consumption of foreign fossil fuels.

Between the late 1970s and 1990s, the National Renewable Energy Labs (NREL) evaluated the economic feasibility of producing biofuels from a variety of aquatic and terrestrial photosynthetic organisms. This work is described in Sheehan et al., “A look back at the U.S. Department of Energy's aquatic species program—biodiesel from algae.” National Renewable Energy Laboratory, Golden, Colo. (1998). Biofuel production from microalgae was determined to have the greatest yield/acre potential of any of the organisms screened. Microalgal biofuel production was estimated to be 8 to 24 fold greater than the best terrestrial biofuel production systems. Current estimates of the potential productivity for algal biofuel production range from 3000 to 10000 gallons/acre. However, a great deal of work still needs to be carried out to optimize the efficient growth of algae, and in particular to grow algae that includes a relatively high amount of lipids.

One method for increasing the amount of lipids produced by algae is to change the growth conditions under which the algae grow. For example, U.S. Patent Publication 2009/0148928 describes the use of a “heterotrophic shift” for increasing lipid production, in which algae or other organisms are first grown under autotrophic conditions, under which the algae grow using photosynthesis in an energy-efficient and cost-effective manner, after which the algae are shifted to heterotrophic growth, where they feed off of supplied fixed carbon sources such as sugar without available sunlight and produce higher lipid levels.

Another method to increase the proportion of lipids in an algal cell culture is to subject the cells to stress, such as nutrient stress. See Rosenberg et al., Curr Opin Biotech 19: 430-436 (2008). Typically this involves bringing cultures to a target concentration, under conditions as near to optimal as possible (i.e., minimal stress and nutrient replete conditions), followed by a sudden cessation of nutrients. This stress effect may also be facilitated by altering salinity, light levels, pH, or temperature. During stress treatment, oleaginous algal species typically divert a greater portion of cellular metabolism toward lipid production, effectively raising cellular lipid concentration (2-3 times) which is then destructively harvested.

Another method to increase lipid accumulation in algae culture is to limit the availability of a specific nutrient, such as nitrogen, phosphate, or silicon, depending on the strain of interest. As with general nutrient stress, once algae growth becomes limited by the unavailability of the depleted nutrient, excess carbon fixed by photosynthesis is shunted towards carbon storage molecules such as starch and lipids. While it is inexpensive to reduce the amount of any specific nutrient in growth media, the net result is likely to be growth rate and yield limiting. One strategy to overcome this limitation is to grow the biomass in a small pond with high nutrient concentrations with fast growth and subsequently transfer the biomass to a larger container while diluting the nutrients to limiting concentrations and induce lipid accumulation. Unfortunately, the solar efficiency of such a strategy is inefficient as biomass growth is only achieved in smaller ponds with lower total productivity thus limiting growth capacity. In addition, the larger volume of dilute media and biomass requires more energy to process. Accordingly, the need remains for methods of more efficiently growing algae, and in particular for efficiently growing algae in a manner that increases their lipid content while keeping processing costs to an economical level.

### SUMMARY OF THE INVENTION

The present invention provides methods of more growing algae such as oleaginous algae more efficiently, either in terms of growing algae so that they produce high levels of lipids for extraction, or in terms of growing the algae in a system where the algae culture is more readily maintained under stable conditions. Accordingly, in one aspect, the present invention provides a method for increasing lipid production by oleaginous algae in an algae culture by inhibiting nutrient assimilation under nutrient replete conditions. Nutrient assimilation can be inhibited by contacting the algae culture with a chemical inhibitor such as chlorate, or nutrient assimilation can be inhibited by transfection of the oleaginous algae with an expression vector including a polynucleotide sequence capable of expressing a nitrate reductase inhibitor and a promoter that is operably linked to the polynucleotide sequence that will express the nitrate reductase inhibitor under controlled conditions.

Another aspect of the invention provides a method for extracting lipid from oleaginous algae, that includes the cycle of subjecting oleaginous algae of an algae culture to stress to increase the lipid content of the oleaginous algae; mixing at least a portion of the algae culture with an lipid-extracting solvent to obtain a solvent-algae mixture; separating the solvent-algae mixture to obtain a solvent-lipid fraction and an extracted algae fraction; and returning the extracted algae fraction to the culture of oleaginous algae, after which the extracted algae fraction and the culture of oleaginous algae are mixed and allowed to grow under non-stressed conditions.

In another aspect, the present invention provides a method for extracting lipid from oleaginous algae that includes the cycle of subjecting oleaginous algae of an algae culture to stress to increase the lipid content of the oleaginous algae; removing at least a portion of the oleaginous algae of the algae culture; extracting a portion of the lipids from the oleaginous algae; and growing the oleaginous algae of the algae culture under non-stressed conditions; wherein the cycle is repeated a plurality of time. When carrying out this method, the lipids can be extracted from the oleaginous algae using a destructive extraction process.

In another aspect, the present invention provides a system for maintaining outdoor algal culture that includes an open artificial algae pond; an algae culture within the open artificial algae pond; and a plurality of buoyant spheres provided in an amount sufficient to substantially decrease the exposed surface area of the open artificial algae pond.

### BRIEF DESCRIPTION OF THE FIGURES

The present invention may be more readily understood by reference to the following drawings wherein:

FIG. 1 provides line graphs showing A) the effect of a variable sized covering of plastic buoyant spheres on water temperatures in a 38 L aquaria illuminated using metal halide illumination, and B) the effect of a variable sized covering of plastic buoyant spheres on evaporation in 38 L aquaria illuminated using metal halide illumination.

To illustrate the invention, several embodiments of the invention will now be described in more detail. Reference will be made to the drawings, which are summarized above. Skilled artisans will recognize the embodiments provided herein have many useful alternatives that fall within the scope of the invention.

### DETAILED DESCRIPTION OF THE INVENTION

The disclosed embodiments of the present invention are in the field of devices, processes, and systems for improved extraction of useful products from cells in culture without loss of culture viability.

### DEFINITIONS

Unless otherwise defined, all technical and scientific terms used herein have the same meaning as commonly understood by one of ordinary skill in the art to which this invention pertains. In case of conflict, the present specification, including definitions, will control.

The terminology as set forth herein is for description of the embodiments only and should not be construed as limiting of the invention as a whole. Unless otherwise specified, “a,” “an,” ““the,” and “at least one” are used interchangeably. Furthermore, as used in the description of the invention and the appended claims, the singular foams “a”, “an”, and “the” are inclusive of their plural forms, unless contraindicated by the context surrounding such. The singular “alga” is likewise intended to be inclusive of the plural “algae.”

The terms “comprising” and variations thereof do not have a limiting meaning where these terms appear in the description and claims.

Also herein, the recitations of numerical ranges by endpoints include all numbers subsumed within that range (e.g., 1 to 5 includes 1, 1.5, 2, 2.75, 3, 3.80, 4, 5, etc.).

The term biocompatible, as used herein, refers to a material that will not adversely affect the growth of algae in the algae culture if the material is material is left in contact with the algae culture for an extended period.

One aspect of the present invention provides methods for efficiently growing algae in a manner that increases their lipid content. In one method, the algae are subject to stress to increase their lipid content, and then subject to non-destructive extraction of the lipid from the algae. More specifically a method for extracting lipid from oleaginous algae is provided that includes a cycle of steps. These steps can be repeated in some embodiments, or in other embodiments a single cycle can be carried out. The cycle includes the steps of subjecting oleaginous algae in an algae culture to stress to increase the lipid content of the oleaginous algae; mixing at least a portion of the algae culture with an lipid-extracting solvent to obtain a solvent-algae mixture; separating the solvent-algae mixture to obtain a solvent-lipid fraction and an extracted algae fraction; and returning the extracted algae fraction to the culture of oleaginous algae. The extracted algae fraction mixes back into the algae culture to form a part of that culture and is allowed to grow under nutrient replete conditions, which replenishes the lipid content of the extracted algae.

The present invention refers to both oleaginous algae and an algae culture. An oleaginous alga is an algae species that can, under known conditions, accumulate a significant portion of its biomass as lipid. For example, embodiments of oleaginous algae are algae species that are capable of accumulating at least 10%, at least 20%, at least 30%, at least 40%, or at least 50% of their biomass as lipid. Suitable oleaginous algae species can be found in the Bacillariophyceae, Chlorophyceae, Cyanophyceae, Xanthophyceaei, Chrysophyceae, Chlorella, Crypthecodinium, Schizocytrium, Nannochloropsis, Ulkenia, Dunaliella, Cyclotella, Navicula, Nitzschia, Cyclotella, Phaeodactylum, and Thaustochytrid classes and genera. A preferred genus of oleaginous algae is Chlorella, which includes numerous species capable of accumulating about 55% of their total biomass as lipids. See for example Miao & Wu, Journal of Biotechnology, 110, p. 85-93 (2004). Suitable Chlorella species include Chlorella vulgaris, Chlorella protothecoides, Chlorella sorokiniana, and Chlorella kessleri.

The algae species used form a part of an algae culture. An algae culture refers to one or more algae species living in an environment that enables their survival and possible growth. The culture conditions required for various algae species are known to those skilled in the art. Examples of the components of an algae culture include water, carbon dioxide, minerals and light. However, the components of an algae culture can vary depending on the algae species, and whether or not conditions for autotrophic or heterotrophic growth are desired. For autotrophic growth, the algae culture will require CO₂ and light energy (e.g., sunlight), whereas heterotrophic growth requires organic substrates such as sugar for the growth of the algae culture, and can be carried out in the absence of light energy. An algae culture requires that appropriate temperature conditions be maintained, and preferably that the culture is mixed to provide even access to nutrients and/or light. While algae can grow in non-aqueous environments, algae culture as referred to herein is algae culture in an aqueous environment, and is therefore a liquid. Preferably the algae culture is a monoculture including a single algae species, or at least is intended as such, taking into account possible contaminating predators and competitors. Use of a monoculture makes it easier to provide optimal culture conditions, and can simplify growing and processing the algae in other ways. However, embodiments of the invention include algae cultures that have more than one species of algae present.

The cycle for increasing the lipid content and extracting the lipid from oleaginous algae includes subjecting oleaginous algae in an algae culture to stress to increase the lipid content of the oleaginous algae. Several mechanisms exist for inducing lipid accumulation in algal cultures. For example, the oleaginous algae will accumulate increased amounts of lipids if subjected to nutrient stress. Nutrient stress is a condition in which insufficient nutrients are available for the algae to freely proliferate, and typically will result in a decrease in the growth rate of the stressed algae species. Nutrient stress can result from a general unavailability or insufficient quantities of a variety of different nutrients, or it can occur as a result of the absence of a single essential nutrient. Examples of essential algae nutrients include carbon dioxide, nitrogen, sulfur, molybdenum, magnesium, specific vitamins, and iron. A preferred nutrient that can be removed to induce nutrient stress is nitrogen. While nutrient deficiency (e.g. nitrogen deficiency) inhibits the cell cycle and production of most algal cellular components, the rate of lipid synthesis remains relatively high, leading to the accumulation of lipids in nutrient-starved algae cells. However, while algae can be stressed by nutrient stress, other types of stress can be applied as well. Essentially, any significant deviation from the preferred algae culture system for an alga species can result in stress. Other sources of stress include too much or too little light, or significant deviations in temperature, pH, or salinity from those preferred by the alga species. The stress should be applied for a number of hours in order to achieve the desired effect of increasing lipid levels. For example, the stress can be applied for a period of about 6 to about 48 hours, for about 10 to 32 hours, or for about 12 to 24 hours. In some embodiments, the algae are stressed by inhibiting nutrient assimilation under nutrient replete conditions, in which nutrients are available but their uptake or use is prevented. This can be carried out, for example, by culturing the algae together with chemical inhibitors, as will be described further herein.

The purpose of stressing the oleaginous algae is to increase their production of lipids. Lipid production can be increased by varying amounts depending on the algae and the stress applied. As noted herein, the lipid context of oleaginous algae can be increased to 10%, 20%, 30%, 40%, or even 50% of the cells dry weight through the application of stress. Lipids, as defined herein, include naturally occurring fats, waxes, sterols, monoglycerides, diglycerides, triglycerides, and phospholipids. The preferred lipids are fatty acid lipids found in triacylglycerides. Free fatty acids are synthesized in algae through a biochemical process involving various enzymes such as trans-enoyl-acyl carrier protein (ACP), 3-hydroxyacyl-ACP. 3-ketoacyl-ACP, and acyl-ACO. Examples of free fatty acids include fatty acids having a chain length from 14 to 20, with varying degrees of unsaturation. A variety of lipid-derived compounds can also be useful as biofuel and may be extracted from oleaginous algae. These include isoprenoids, straight chain alkanes, and long and short chain alcohols, which short chain alcohols including ethanol, butanol, and isopropanol.

Subsequent to increasing the lipid levels in the oleaginous algae by applying stress, at least a portion of the algae culture are mixed with a lipid-extracting solvent to obtain a solvent-algae mixture, after which the mixture of the lipid-extracting solvent and the oleaginous algae are separated to obtain a solvent-lipid fraction and an extracted algae fraction. Typically, only portions of the algae culture are removed to reduce the amount of lipid-extracting solvent required, to increase the effectiveness of extraction, and to minimize the additional stress on the oleaginous algae. The portions may be obtained in a continuous or non-continuous fashion, and in some embodiments the entire algae culture may be subjected to extraction. This extraction process is described in greater detail in U.S. Patent Publication No. 20090181438, which is incorporated herein by reference. To summarize, the cells are combined with a lipid-extracting solvent for a number of minutes in a process which has no significant effect on cell survivability. For example, the cells can be combined with a lipid extracting solvent for about 5 minutes. While the algae cells are in the lipid extracting solvent, it may also be preferable to briefly sonicate the algae cells (e.g., for a few seconds) at a frequency from about 20 kHz to 1 MHz, with frequencies of 20 KHz to 60 KHz being preferred.

A lipid-extracting solvent is an organic solvent that will take up lipids from oleaginous algae that are immersed in the solvent. Examples of lipid-extracting solvents include hydrocarbons with a length from C₄ to C₁₆, with hydrocarbons having a length of C₁₀ to C₁₆ being preferred. Examples of suitable lipid-extracting solvents include 1,12-dodecanedioic acid diethyl ether, n-hexane, n-heptane, n-octane, n-dodecane, dodecyl acetate, decane, dihexyl ether, isopar, 1-dodecanol, 1-octanol, butyoxyethoxyehteane, 3-octanone, cyclic paraffins, varsol, isoparaffins, branched alkanes, oleyl alcohol, dihecylether, and 2-dodecane. Oleaginous algae that have had their lipids removed by extraction are referred to herein as extracted algae.

Once the lipids have been extracted from the oleaginous algae, the lipid-extracting solvent and the oleaginous algae are separated to obtain a solvent-lipid fraction and an extracted algae fraction. This can be carried out in a phase-separation chamber, in which the aqueous solution including the extracted algae separates from the lipid-including lipid-extracting organic solvent. Because the solvents have different densities and affinities, they will naturally separate, with the aqueous solution typically foaming a lower layer, while the organic solvent forms an upper layer. While this separation occurs naturally, it can be facilitated by applying sonication to decrease haze and emulsion formation. The solvent including the extracted lipid can then be collected from the phase separation chamber, and the solvent further processed (e.g., by distillation) to isolate and collect the lipid obtained.

The extracted algae fraction is typically then returned to the culture of oleaginous algae. The extracted algae fraction mixes back into the algae culture to form a part of that culture and is allowed to grow under non-stressed (e.g., nutrient replete) conditions, which allow the extracted algae to grow. Non-stressed refers to the normal culture conditions used for culturing the algae, in which the stress that was earlier applied is absent. An example of non-stressed conditions is nutrient replete conditions, in which all of the nutrients that are normally used by the algae are available in sufficient quantities that the algae are not subject to stress and will grow at their normal rate. Interestingly, extraction of the algae has been shown to extend culture growth times from one to more than five weeks under some conditions, which may be associated with the partitioning of waste products during the extraction. However, in the reverse situation, in which extraction has a somewhat detrimental effect on the algae, it may be preferable to carry out the extraction on only a fraction (e.g., 10-50%) of the overall algae culture in order to provide the algae culture with an opportunity to rebound and replace damaged cells during the non-stress period. Preferably, the oleaginous algae are allowed to grown under non-stressed conditions for about 12 to about 48 hours, though in other embodiments the algae may be allowed to grow for from 24 to 72 hours, or from 6 to 24 hours.

Augmenting Algal Culture Lipid Production with Pulsed Stress

Given that the stress response is typically rapid (taking less than 24 h), and normal growth can recover after the removal of stress (again taking less than 24 h), an advantageous strategy to maximize algal lipid production is to apply pulsed stress induction methods on cultures growing continuously under a non-destructive extraction process, as described above. Because the lipids are not normally completely extracted, and for other reasons, pulsed stress allows high lipid levels to be achieved more rapidly relative to beginning with a new algae culture. Accordingly, in some embodiments of the invention, the cycle that includes stressing the algae, extracting the lipids, and then allowing the algae to grow under non-stressed conditions is repeated a plurality of times. For example, nutrients can be withheld for a set period of time (e.g., 6-48 hours), or until a set lipid concentration or critical cell concentration is reached. During or after this stress period, lipids can be continuously removed non-destructively in vivo using the extraction process, but at much higher yields due to the induction of higher lipid levels. Cessation of stress periods would be brief (12-48 h) maximizing lipid induction periods. Suitable periods for conducting stress and periods for recovery from stress can be determined by monitoring the oleaginous algae cells, for example by analyzing their lipid levels. Less stable cultures could also be pulse stressed to milder set points, with or without the non-destructive extraction process, resuming in vivo lipid harvest upon release of stress, followed by a variable (24-72 h) non-stress recovery period allowing culture stabilization.

Cycling the algae through periods of non-stress followed by periods of stress have the advantage of allowing an algae culture that has developed a high lipid content to be maintained. Accordingly, cycling may be useful together with other forms of lipid extraction, such as destructive extraction. The present invention provides a method for extracting lipid from oleaginous algae that includes the cycle of subjecting oleaginous algae of an algae culture to stress to increase the lipid content of the oleaginous algae; removing at least a portion of the oleaginous algae of the algae culture; extracting a portion of the lipids from the oleaginous algae; growing the oleaginous algae of the algae culture under non-stressed conditions; wherein the cycle is repeated a plurality of time.

Pulsed stress can be used to increase lipid levels with either non-destructive or destructive extraction methods. When destructive extraction is used, only a portion of the algae culture is typically removed, to allow the remaining algae culture to continue to be cycled through non-stress and stress. A destruction extraction process is a process in which the algae are killed by the process of extracting the algae, unlike the non-destructive lipid extraction process described herein. Destructive algae harvesting can include treatment of oleaginous algae in algae culture, or it can include the treatment of dried biomass obtained from an algae culture. Methods for the destructive extraction of algae are described by Grima et al., Biotechnology Advances, 20, p. 491-515 (2003).

Data have been obtained using the non-destructive extraction process on Nannochloropsis algae grown in indoor 800 L photobioreactors. Photobioreactors subjected to continuous NDEP and pulse fed nutrients 24 hours after the nitrate/nitrite concentrations were undetectable (using test strips) recovered approximately two times the lipid levels as compared to an identical photobioreactor kept replete in nutrients. See Table 1, which shows the effect of culture nutrition on the amounts (mg/L) of extractable algal lipids using a normal (1:1 solvent to culture, 1× pass ultrasonic/static mixed) NDEP and an aggressive (1:1 solvent to culture, 5× pass, ultrasonic/static mixer) NDEP.

TABLE 1

| | Nutrient Replete | Nutrient Stressed | |
|---|---|---|---|
| | Culture | Culture (24 h | |
| | (nitrate added daily) | post nitrate = 0) | Difference |
| Normal (1x) NDEP | 0.037 ml/L | 0.086 ml/L | 2.32x |
| Aggressive (5x) | 0.043 ml/L | 0.096 ml/L | 2.23x |
| NDEP | | | |

### Artificial Induction of Nutrient Limitation Through Inhibition of Nutrient Assimilatory Processes.

One of the problems with using stress such as nutrient stress to increase lipid production is that it can be difficult to suddenly change the nature of an algae culture from being non-stressed to stressed, or vice-versa. To overcome these limitations a strategy to induce nutrient limitation without actually changing the nutrient concentration has been developed. Accordingly, another aspect of the invention provides a method for increasing lipid production by oleaginous algae in an algae culture by inhibiting nutrient assimilation under nutrient replete conditions. Using this method, nutrient assimilation can be inhibited by contacting the algae culture with an inhibitor that prevents the uptake of an essential nutrient. Alternately, the oleaginous algae species can be genetically modified to inducibly express proteins that limit the rate at which nutrients can be assimilated into biomass. This can be achieved by targeting any one of several cellular processes such as nutrient import, modification (e.g. oxidation, reduction), or incorporation.

### Chemical Induction of Nutrient Stress to Enhance Lipid Yields

In one embodiment of the invention, nutrient assimilation is inhibited by contacting the algae culture with a chemical inhibitor. It is generally known that algal lipid content can increase when the cultures are placed under nutrient stress (e.g., nitrogen deprivation). The use of chemical inhibitors or competitors for one or more nutrients used by the cells can “trick” the cells into “thinking” they are under nutrient stress, thereby inducing the cells to increase lipid production even though the algae cells are in the presence of excess or sufficient (i.e., replete) nutrients. One example of a chemical inhibitor that is able to simulate nutrient stress in algae cells is chlorate (ClO₃), which mimics nitrate in the metabolic system. Chlorate rapidly inhibits nitrate uptake at very low micromolar concentrations. This, in turn, can rapidly induce lipid accumulation in the algae, as the effect of inhibiting nitrate uptake mimics nutrient stress caused by a lack of nitrate in algae. Chlorate-stressed algae may up-regulate high affinity nitrate transporters, but they will still be blocked from utilizing nitrate. If added at late log phase, chlorate will cause lipid accumulation at the exclusion of further growth, boosting overall lipid % per cell.

Chlorate or other chemical inhibitors could be added to algae in continuous manner or the inhibitor can be added to a separate algae pond, referred to herein as an algae stress pond, that is filled using a slip stream from algae ponds where active growth of oleaginous algae is occurring. This would lead to a reduced residence time in the algae stress pond due the immediate chemical response which is faster than the induction of lipid formation caused by normal metabolically induced stress. Chlorate stress can be removed by adding ammonia or ammonia salt to an algae culture.

Chlorate or other chemical inhibitors can also be added to a terminal stream of algae culture that is being switched to heterotrophic growth conditions to induce a more rapid boost of the lipid content. Chlorate also provides the advantage of being anti-microbial and will reduce bacterial competition with the algae for fixed carbon added in any heterotrophic production of additional lipids by the algae. Using metabolic inhibitors for induction of nutrient stress should raise levels of lipids produced per unit biomass and reduce loss of nutrients/sugars to other organisms and biomass.

Alternative inhibitors of other specific essential nutrients such as sulfur, molybdenum, magnesium, specific vitamins, iron, and the like could also be used for such a purpose, and are known to those skilled in the art. For instance sulfur stress could be induced by the specific sulphate update inhibitor selanate (Honda et al., J Plant Nutr. 21, p. 601-614 (1998)). Aluminum compounds can be used as inhibitors of magnesium uptake and could be utilized in a similar fashion (Tani et al., Appl Micro Biotech 65, p. 344-348 (2003)).

Genetic Modification to Provide Proteins that Will Simulate Nutrient Stress Upon Expression

In another aspect of the present invention, nutrient assimilation is inhibited by transfection of oleaginous algae with an expression vector including a polynucleotide sequence capable of expressing a nitrate reductase inhibitor and a promoter that is operably linked to the polynucleotide sequence that will express the nitrate reductase inhibitor under controlled conditions. In some embodiments, the oleaginous algae is a species of Chlorella, whereas in other embodiments the oleaginous algae is Chlorella vulgaris. A polynucleotide sequence capable of expressing the nitrate reductase inhibitor is described by GenBank Accession Number AB032413.1, as well as homologs thereof.

One means to limit nitrogen availability is through the utilization of the nitrate reductase inhibitor (NRI) found in higher plants. Nitrate reductase (NR) is required to reduce nitrate to nitrite and subsequently further reduced to ammonia which is assimilated into biomass. In the absence of media supplementation with ammonia, the only source for nitrogen assimilation for green algae (e.g., oleaginous algae) is through the reduction of nitrate.

Methods for the transfection of various types of algae are known to those skilled in the art. See for example Radakovits et al., Eukaryotic Cell, 9, 486-501 (2010), which is incorporated herein by reference. A variety of methods have been used to transfer DNA into microalgal cells, including agitation in the presence of glass beads or silicon carbide whiskers, electroporation, biolostic microparticle bombardment, and Agrobacterium tumefaciens-mediated gene transfer.

The term “operably linked” refers to the arrangement of various polynucleotide elements relative to each other such that the elements are functionally connected and are able to interact with each other. Such elements may include, without limitation, a promoter, an enhancer, a polyadenylation sequence, one or more introns and/or exons, and a coding sequence of a gene of interest to be expressed. The nucleic acid sequence elements, when operably linked, act together to modulate the activity of one another, and will affect the level of expression of the gene of interest (i.e., the nitrate reductase inhibitor gene). By modulate is meant increasing, decreasing, or maintaining the level of activity of a particular element. The position of each element relative to other elements may be expressed in terms of the 5′ terminus and the 3′ terminus of each element.

The term “transfection” is used to refer to the uptake of foreign DNA by a eukaryotic cell such as an algae cell. A cell has been “transfected” when exogenous DNA has been introduced inside the cell membrane. The term refers to both stable and transient uptake of the genetic material.

The term “expression vector” refers to any type of genetic construct comprising a nucleic acid coding for a RNA capable of being transcribed. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well.

The term “promoter” refers to a nucleic acid sequence that regulates, either directly or indirectly, the transcription of a corresponding nucleic acid coding sequence to which it is operably linked. The promoter may function alone to regulate transcription, or, in some cases, may act in concert with one or more other regulatory sequences such as an enhancer or silencer to regulate transcription of the transgene. The promoter comprises a DNA regulatory sequence, wherein the regulatory sequence is derived from a gene, which is capable of binding RNA polymerase and initiating transcription of a downstream (3′-direction) coding sequence.

A promoter generally comprises a sequence that functions to position the start site for RNA synthesis. The best-known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Alternatively, certain advantages may be gained by positioning the coding nucleic acid segment under the control of a recombinant or heterologous promoter, which refers to a promoter that is not normally associated with a nucleic acid sequence in its natural environment. Such promoters may include promoters of other genes, and promoters isolated from any other virus, or prokaryotic or eukaryotic cell, and promoters not “naturally occurring,” i.e., containing different elements of different transcriptional regulatory regions, and/or mutations that alter expression. Furthermore, it is contemplated the control sequences that direct transcription and/or expression of sequences within non-nuclear organelles such as mitochondria, chloroplasts, and the like, can be employed as well. Expression under controlled conditions refers to activation of the promoter in response to a specific condition in the algae culture, so that nutrient stress can be prompted when desired. Conditions useful for prompting activation of the promoter are further described herein.

The determination of percent identity or homology between two sequences is accomplished using the algorithm of Karlin and Altschul (1990) Proc. Natl. Acad. Sci. USA 87: 2264-2268, modified as in Karlin and Altschul (1993) Proc. Natl. Acad. Sci. USA 90:5873-5877. Such an algorithm is incorporated into the NBLAST and)(BLAST programs of Altschul et al. (1990) J. MoI. Biol. 215:403-410. BLAST nucleotide searches are performed with the NBLAST program, score=100, wordlength=12 to obtain nucleotide sequences homologous to the nucleic acid molecules of the invention. BLAST protein searches are performed with the)(BLAST program, score=50, wordlength=3 to obtain amino acid sequences homologous to the protein molecules of the invention. To obtain gapped alignments for comparison purposes, Gapped BLAST is utilized as described in Altschul et al. (1997) Nucleic Acids Res. 25: 3389-3402. When utilizing BLAST and Gapped BLAST programs, the default parameters of the respective programs (e.g., XBLAST and NBLAST) are used.

The Nitrate Reductase Inhibitors (NRI) purified from corn, rice, spinach, Arabidopsis, and soybean have been shown to inactivate NR activity (Solomonson et al., J Biol Chem 261(24):11230-11294 (1986); Jolly & Tolbert, Plant Physiol. 62, p. 197-203, (1978); Yamaya & Solomonson, Plant Physiol, 65, p. 146-150 (1980)). In the presence of NRI, the NR enzymes ability to oxidize NADH and reduce Nitrate has been lost. In a plant, NRI is required to coordinate NR activity with photosynthetic processes including the opening and closing of stomata. In algae this control is not necessary, thus it is unlikely that an inhibitor of NR would be present and to date only a single distant homologue to spinach NRI has been found in green algae. The introduction and expression of a compatible NRI gene into green algae could inactivate the native algal NR complex and reduce Nitrogen bioavailability thus mimicking nutrient limitation. Of important note, Yamaya & Solomonson demonstrated that purified NRI extract from corn effectively inactivated the Chlorella vulgaris NR activity in vitro.

While not intending to be bound by theory, the NRI protein appears to act by proteolytically cleaving the NR complex leaving it unable to reduce Nitrate (Yamaya &, Boesel, Plant Physiol. 65, p. 141-145 (1980)). In all studies to date, the NADH:Nitrate reductase activity is inactivated by the presence of NRI or a similar protein extract.

While the protein complex for NRI has been purified from several sources, only one gene has been identified to date to encode a functional NRI protein (Sonoda et al., Planta, 216, p. 961-8. (2003)). The spinach NRI gene was identified through the PCR amplification of cDNA using degenerate primers designed from the terminal sequence of the purified protein. Homologous genes to NRI in spinach can be found in many plants including Arabidopsis thaliana, rice, poplar, and the moss Physcomitrella patensis.

Two components are necessary for the strategy of artificially induced nutrient limitation to be a successful inducer of lipid accumulation on a large scale: 1) the induced protein (NRI) must in fact inactivate the NR complex and reduce the bioavailable nitrogen and 2) the NRI protein must be tightly regulated and inexpensively induced (i.e., expressed under controlled conditions). To address the issue of targeted inactivation of the algal nitrate reductase a variety of NRI homologs could be screened for NRI activity using an expression vector using the constitutively active actin promoter to drive the expression of the target NRI gene and selection for growth only on reduced nitrogen sources. The source(s) of NRI genes will likely include a variety of plants; however homologs to the spinach NRI are found across the eukaryotic kingdom. In fact it is likely that nitrate reductase inhibitors which are of a different evolutionary origin and are not homologous to the spinach, corn, or rice NRI may be of use and may come from bacteria or archaea. As such the target NRI (by function) is not limited to homologs of the NRI identified in spinach.

During algae production, constitutive expression of NRI is not desirable. If the NRI protein is expressed at the wrong time it may prematurely limit growth and slow production. To reduce this risk, a strong repression must be maintained until it is released by an activator at the appropriate time and duration. In order for this process to become economically feasible an induction system which is inexpensive to apply on a large scale should be used. Induction can be either through low dose chemical treatment or through physical treatment such as pH shift, temperature shift, pressure shift, photoinduction, etc. The development of an induction system will require a genetic promoter which is tightly controlled in algae, but induced by low specific signal molecule concentration. An example of such a system is detailed in the work by Kato et al. (J. of Biosci and Bioeng., 104, p. 207-213, (2007)) where transgene expression is controlled by the well characterized lac regulation system derived from Escherichia coli. In this case a traditional expression vector for Chlamydomonas chloroplast gene expression was modified to add lac protein binding motifs. In the absence of an inducer molecule (e.g., IPTG) the expression of the target gene(s) is repressed. Upon addition of the inducer the lac protein releases from the promoter region and allows full expression of the targeted gene. Specifically two lac proteins bind to neighboring lac binding sites in the promoter forming a homo-dimer that creates a loop structure in the DNA preventing the processing of the RNA polymerase complex. The inducer molecule binds to the lac protein which releases from the DNA and the looped DNA promoter region is free for normal unrepressed expression. Similar but alternative inducible systems are already characterized and may be derived again for many species. Some examples of other well characterized signal molecule/gene regulatory mechanism include but are not limited to nitrous oxide, copper or other heavy metals, light quality and light intensity. Additionally, it may be necessary to tag the protein for targeted degradation. This will give the protein a high turnover rate so that in the absence of the inducer system the protein product will become inactive and normal growth can resume if required.

The induction of artificial nutrient limitation preferably occurs at one of three locations in a production plant. First, the algae can be constitutively induced at low levels to maintain a moderate constant nutrient deprivation without complete starvation. This will allow the algae to grow and maintain a higher lipid content than normal cells but at a loss of optimal growth rate. Second, after a pond or multiple ponds grow to maturity and maintain a steady state growth a slip stream can be pulled off to a holding pond where the artificial nutrient limitation is induced without dilution or further processing. Finally, the heterotrophic boost process can be combined with the artificial nutrient limitation. Heterotrophic boost is switching the algae culture to heterotrophic conditions prior to lipid extraction in order to increase lipid levels. Under conditions of nitrogen limitation and excess reduced carbon availability, lipid storage would increase rapidly. In any of the above scenarios, after obtaining the optimal lipid content, the biomass is then delivered for lipid extraction using processes such as but not limited to the non-destructive lipid extraction process described herein.

### Improved Maintenance of Outdoor Algae Cultures Using Buoyant Spheres

Methods of increasing the lipid content of oleaginous algae are an important aspect of increasing the efficiency of obtaining biofuels from algae culture. Another important aspect of increasing the efficiency of obtaining biofuels from algae culture is increasing the ability to easily maintain a healthy oleaginous algae culture, particularly when the algae are being repeatedly subjected to non-destructive lipid extraction. This issue is particularly important for outdoor algae cultures such as those grown in open ponds, which do not typically provide much control over temperature and lighting and are vulnerable to contamination by other microorganisms, such as other algal species or bacteria.

Pond evaporation in traditionally shallow (e.g., less than 15 cm deep) outdoor algal cultures represent a significant water loss, which can reach 10% of pond volume per day. Evaporative water loss is influenced by surface wind speeds and warmer temperatures which effectively increase the salt concentrations of ponds, and in sensitive species, disrupt lipid production or culture stability. Procuring a pure water source to replace water lost is a typical mitigation method. However, access to a pure water source can be difficult to obtain (e.g., it may require a permit), or it can be expensive (e.g., using reverse osmosis).

Another factor involved in the maintenance of outdoor ponds is temperature control. Heat loss in cool climates or during night-periods in arid climates can significantly reduce pond temperatures. Conversely, extensive periods of sunlight in warmer climates can significantly increase pond temperatures. Although heating or cooling of small outdoor ponds can be done without excessive expense, it is not economically attractive to provide heating or cooling for a larger shallow ponds (e.g., >10 m²). Since many algal species are sensitive to temperatures, salinity and light, operational tools that can be readily deployed to help maintain proper growth conditions are needed.

One such operational tool is buoyant spheres, which can be effective for both reducing heat exchange and evaporation in outdoor algae culture. Accordingly, one aspect of the invention provides a system for maintaining outdoor algal culture that includes an open artificial algae pond, an algae culture within the open artificial algae pond; and a plurality of buoyant spheres provided in an amount sufficient to substantially decrease the exposed surface area of the open artificial algae pond.

An outdoor algae culture is an algae culture that is positioned in an environment where it is exposed to the elements. In other words, it is “outdoors” rather than “indoors,” in the traditional sense that these words are used. By outdoor, it is not meant that the algae culture be provided in a rural setting. For example, an outdoor algae culture can be found within an urban setting, or even associated with a building, such as a rooftop algae culture.

A pond is a body of standing water that is smaller than a lake. To retain standing water, the pond must include a basin that can hold a significant quantity of water. The maximum size of a pond, as defined herein, is about 20,000 square meters. An open pond is one that is open, at least in part, to the elements, for the majority of the time. For example, the mere fact that a pond may occasionally be covered at certain times, such as with a tarpaulin to prevent excessive contamination, does not render the pond “non-open” as the term is used herein. The provisional of walls for a pond would also not prevent it from being open, so long as it retained exposure to the elements through the roof for the majority of the time. However, for a pond to be open, it does need to be exposed to the elements to a sufficient degree that the elements will have an effect on the pond, and in particular the algae culture in the pond. For example, a pond that is enclosed within a structure that is merely not airtight, but prevents significant contact with the elements, would not be an open pond.

An artificial algae pond is a man-made structure that has been built for the express purpose of farming algae (i.e., algaculture). An artificial algae pond is typically fairly shallow to allow light to reach the majority of the algae within the pond, and typically has a consistent depth to provide the maximum area for growth within the zone that is accessible to light. An artificial algae pond will also typically include a relatively waterproof material either coating or making up the sides of the pond to decrease water loss through the sides of the pond, and prevent exterior matter from entering into the pond through the sides of the pond.

In a preferred embodiment, the open artificial algae pond has a surface area greater than 10 square meters. While buoyant spheres can be used for smaller open artificial algae ponds, smaller ponds generally have lower maintenance costs and therefore the benefit of using the buoyant spheres is decreased. Likewise, open artificial algae ponds having an even larger surface area may provide greater benefits. For example, in other embodiments, the open artificial algae pond can have a surface area greater than 50 square meters, greater than 100 square meters, or greater than 1000 square meters.

Open artificial algae ponds come in various configurations. For example, in some embodiments of the invention, the open artificial algae pond is a raceway-type pond. Raceway-type algae ponds are known to those skilled in the art. In brief, a raceway-type pond is divided into a rectangular grid, with each rectangle containing one channel in the shape of an oval, like an automotive raceway circuit. Each rectangle also typically includes a paddlewheel to provide continuous water flow around the circuit

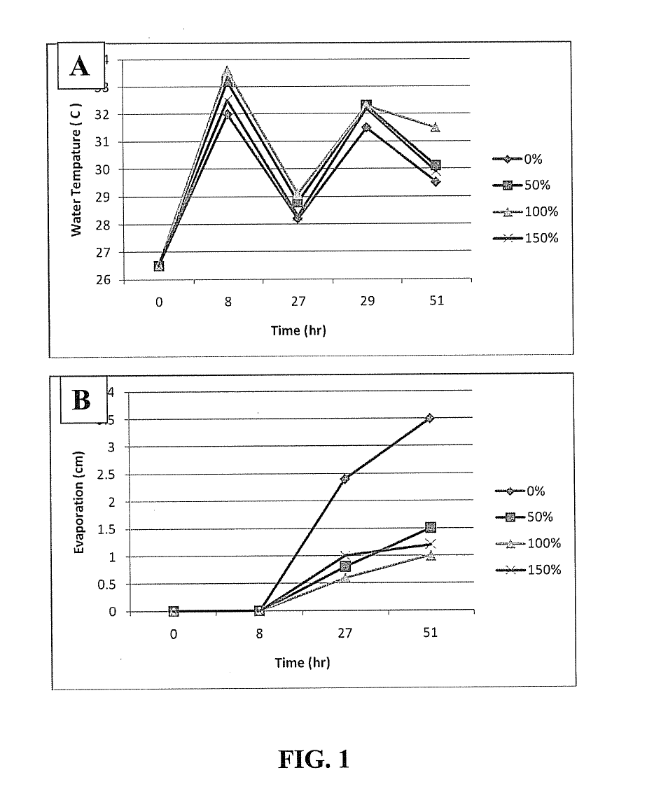

In order to maintain the outdoor algae culture in the open artificial algae pond, a plurality of buoyant spheres is provided. By covering a significant portion of the surface of the algae pond, the buoyant spheres are able to reducing heat exchange and/or evaporation from the algae pond to the outside environment. See for example FIG. 1, which shows the effects of buoyant spheres covering various percentages of the surface of an artificial algae pool on water temperatures and the rate of evaporation over time. Metal halide light was used in the experiments carried out to prepare FIG. 1, because it simulates both the heat and light of natural sunlight. Further, the 150% level of buoyant spheres shown in the figure represents an amount of spheres sufficient to create one and a half layers of buoyant spheres over the surface of the algae pond. In addition to maintaining temperature and water levels, the buoyant spheres can also provide other benefits that help maintain an algae culture, such as decreasing the out-gassing of added carbon dioxide from the open artificial algae pond. The buoyant spheres have an overall density that is lower than that of the liquid in the algae pond, so that the weight of the liquid displaced by the buoyant spheres is sufficient to raise the buoyant spheres to the surface of the algae pond.

The buoyant spheres should be relatively small in size to facilitate their handling and use in the algae ponds. For example, embodiments of the invention can include buoyant spheres that have a diameter ranging from 0.1 to 50 cm, from 0.1 to 15 cm, or from 0.5 to 5 cm. While referred to herein as spheres, it should be noted that it is not necessary for the buoyant spheres to be perfectly spherical in shape in order to function. In the context of the present invention, spherical shapes have the advantage of forming a permeable layer when placed on a surface, such as the surface of an algae pond. This is because the spheres cannot fit tightly together, but rather will leave gaps at the intersections between various spheres. However, there are a wide variety of other shapes that also can be placed on a surface that do not tightly fit together, but instead leave gaps. It is preferable for there to be gaps in the layer formed by the spheres since this will allow gasses used and generated by the algae (e.g., carbon dioxide and oxygen) to pass through the surface of the algal pond. Other suitable “buoyant spheres” include buoyant oval-shaped or ovoid objects.

The buoyant spheres can be formed from a variety of biocompatible, low-density materials. Low-density, as used herein, refers to materials with a density less that of the algae culture. For example, the buoyant spheres can be made from low-density polypropylene or other relatively low density biocompatible plastics. In addition, the buoyant spheres can be hollow spheres. If hollow spheres are used, the spheres can be manufactured from heavier materials such as glass, so long as the overall density of the object is kept lower than that of the liquid provided in the algae pond (e.g., water). If hollow spheres are used, the hollow cavity within the sphere can be filled with air or a desired gas or mix of gasses (e.g., argon and nitrogen) to further insulate the algae pond and reduce heat exchange.

Buoyant spheres could also include light wavelength shifting materials to facilitate algal photosynthesis and growth. The light wavelength shifting materials can be incorporated into the material of the spheres, or placed within the cavity of hollow spheres. Buoyant spheres including light wavelength shifting materials should be transparent to allow light to reach the shifting materials. Light wavelength shifting materials are materials that absorb light in a wavelength that cannot be used to carry out photosynthesis by the algae, such as a wavelength from about 400 to 600 nanometers, and shifting the wavelength of that light into a range that can be used by the algae to carry out photosynthesis, such as 650 to 680 nanometers. See for example the use of accessory pigments in U.S. Patent Publication 20090181438, the disclosure of which is incorporated by reference. The buoyant spheres can be made from transparent or clear materials to increase the passive heating of water by a greenhouse effect during cooler periods, or made from opaque materials to reduce passive heating and light penetration during sunny periods.

In order to be effective, the buoyant spheres should be provided in an amount sufficient to substantially decrease the exposed surface area of the open artificial algae pond. The number of spheres that should be provided will vary depending on the size of the buoyant spheres and the size of the open artificial algae pond. The exposed surface area is the portion of the surface of the artificial algae pond that is not in contact with a buoyant sphere. An amount sufficient to substantially decrease the exposed surface area is an amount sufficient to create a significant decrease in temperature exchange or the rate of evaporation. For example, an algae pool having a rate of evaporation of greater than 2 cm/day is too high, and thus sufficient buoyant spheres should be added to result in a rate of evaporation of less than 1 cm per day. Likewise, open algae pond can lose more than 20° C. on a cold night, which is too great a loss of heat. Again, sufficient buoyant spheres should be added to result in a temperature change of less than 10° C.

For example, in different embodiments of the invention, the buoyant spheres may be present on at least 25% of the surface of the open artificial algae pond, whereas in other embodiments they may be present on at least 50% of the surface, at least 75% of the surface, or 100% of the surface. Note that these percentages regard to the percentage of the surface of the pond that includes buoyant spheres, and that this is different from the amount of surface area that is in contact with the buoyant spheres. While the amount of surface area that is in contact with the buoyant spheres is necessarily less than 100%, the amount of surface that includes the buoyant spheres can be 100%, in which case the spheres would cover the entirety of the surface of the pond, yet would leave gaps between the spheres. In some embodiments of the invention, the number of buoyant spheres provided is in excess of the amount needed to cover 100% of the surface of the algae pond, resulting in the formation of an additional layer or partial layer. In such cases, greater than 100% of the surface of the algae pond is said to be covered with buoyant spheres.

Deployment of buoyant spheres would involve mass release onto open artificial algae ponds and passive self organization of the spheres into a layer through pond currents. Deployment of buoyant spheres would be compatible with raceway paddlewheels and submerged pump systems. Depending on monitored environmental conditions or algal viability, buoyant spheres could be rapidly added or removed on an hourly, daily, or diurnal basis through surface skimmers, screens, augers, paddlewheel buckets etc. Cleaning of the buoyant spheres could be facilitated by weak detergents, acid washes, or ultrasonic fields. Normal gas exchange between air and water would be facilitated by rotational motion of spheres. In seasonal or colder climates buoyant spheres would be attractive alternatives to the use of greenhousing (i.e., micro-greenhousing). The buoyant spheres would have significantly lower capital costs, require less maintenance, easier to remove, are more efficient at passive solar trapping, more robust in regions prone to high snow-loads or high winds.

An example has been included to more clearly describe a particular embodiment of the invention and its associated cost and operational advantages. However, there are a wide variety of other embodiments within the scope of the present invention, which should not be limited to the particular example provided herein.

### EXAMPLE

### Example 1

### Increased Algae Production Using Chlorate Stress

Nannochloropsis sp., a unicellular alga capable of producing large amounts of triacylglycerides, can be grown in f/2 medium with 1/3^{rd} strength Instant Ocean under indoor lighting in 800 L raceway systems either kept moving with a submerged pump or paddlewheel. The algae are first grown under normal, nutrient replete conditions. Upon achieving a set concentration in culture sufficient for economical extraction (e.g., 1.0 g/L), an amount of chlorate sufficient to block nitrogen uptake by the Nannochloropsis is added. The Nannochloropsis culture is then allowed to grow under these conditions of nutrient assimilation inhibition for 18 hours. Twenty five percent of the algae culture is then removed and subjected to a non-destructive extraction process, while ammonia is added to the remaining in an amount sufficient to renew normal nitrogen uptake.

The removed portion of the culture is then mixed with dodecane or undecane or a mixture of the two for 15 minutes, during which sonication is applied for 2 seconds at 40 kHz. The mixture is partitioned with the upper solvent level removed by decanting, and the lower aqueous layer is then returned to the original algae culture, in which non-stressed growth has resumed as a result of the addition of ammonia. The algae culture is then allowed to grow for 36 hours under non-stressed conditions, after which the algae culture is again subjected to stress by addition of chlorate for 18 hours after which one fourth of the culture is extracted using solvent, as described.

The use of pulsed stress in which nutrient assimilation is inhibited by chlorate under nutrient-replete conditions should result in higher levels of lipid obtained during subsequent cycles as compared to an identical system in which the cycle of growth, stress, and harvesting is only carried out once.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

1. A method for increasing lipid production by oleaginous algae in an algae culture by inhibiting nutrient assimilation under nutrient replete conditions.

2. The method of claim 1, wherein nutrient assimilation is inhibited by contacting the algae culture with a chemical inhibitor.

3. The method of claim 2, wherein the chemical inhibitor is chlorate.

4. The method of claim 1, wherein the nutrient is nitrogen.

5. The method of claim 1, wherein nutrient assimilation is inhibited by transfection of the oleaginous algae with an expression vector including a polynucleotide sequence capable of expressing a nitrate reductase inhibitor and a promoter that is operably linked to the polynucleotide sequence that will express the nitrate reductase inhibitor under controlled conditions.

6. The method of claim 1, wherein the oleaginous algae comprises algae of the genus Chlorella.

7. A method for extracting lipid from oleaginous algae, comprising the cycle of subjecting oleaginous algae of an algae culture to stress to increase the lipid content of the oleaginous algae;
mixing at least a portion of the algae culture with an lipid-extracting solvent to obtain a solvent-algae mixture;
separating the solvent-algae mixture to obtain a solvent-lipid fraction and an extracted algae fraction; and returning the extracted algae fraction to the culture of oleaginous algae, and further wherein the extracted algae fraction and the culture of oleaginous algae are mixed and allowed to grow under non-stressed conditions.

8. The method of claim 7, wherein the cycle is repeated a plurality of times.

9. The method of claim 7 wherein the algae culture is subjected to stress for a period of about 6 to about 48 hours.

10. The method of claim 7, wherein the extracted algae fraction and the culture of oleaginous algae are allowed to grow under non-stressed conditions for about 12 to about 48 hours.

11. The method of claim 7, wherein the stress is nutrient stress.

12. The method of claim 7, wherein the algae culture is subjected to stress by inhibiting nutrient assimilation under nutrient replete conditions.

13. A method for extracting lipid from oleaginous algae, comprising the cycle of subjecting oleaginous algae of an algae culture to stress to increase the lipid content of the oleaginous algae;
removing at least a portion of the oleaginous algae of the algae culture;
extracting a portion of the lipids from the oleaginous algae; and
growing the oleaginous algae of the algae culture under non-stressed conditions;
wherein the cycle is repeated a plurality of time.

14. The method of claim 13, wherein the step of extracting a portion of the lipids from the oleaginous algae comprises a destructive extraction process.

15. A system for maintaining outdoor algal culture, comprising:
an open artificial algae pond;
an algae culture within the open artificial algae pond; and
a plurality of buoyant spheres provided in an amount sufficient to substantially decrease the exposed surface area of the open artificial algae pond.

16. The system of claim 15, wherein the open artificial algae pond has a surface area greater than 10 square meters.

17. The system of claim 15, wherein the open artificial algae pond is a raceway-type pond.

18. The system of claim 15, wherein buoyant spheres have a diameter from about 0.1 to about 15 centimeters.

19. The system of claim 15, wherein the buoyant spheres have a hollow interior.

20. The system of claim 15, wherein the buoyant spheres comprise a light wavelength shifting material.

21. The system of claim 15, wherein the buoyant spheres are transparent.

22. The system of claim 15, wherein the buoyant spheres are opaque.

23. The system of claim 15, wherein the buoyant spheres cover at least 50% of the surface of the open artificial algae pond.
